(19) United States
(12) Patent Application Publication
(10) Pub. No.: US 2006/0287706 A1
(43) Pub. Date: Dec. 21, 2006

(54) Stent with mechanically interlocking struts and methods for making the same

(76) Inventors: Daniel Olsen, Wharton, NJ (US); Jay Mason, Easton, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/153,637

(22) Filed: Jun. 15, 2005

(51) Int. Cl. A61F 2/06

(52) U.S. Cl. 623/1.15

(57) ABSTRACT

A stent having mechanically interlocked strut sections and methods of making the same. Sets of longitudinally adjacent strut sections have closed ends that are mechanically interlocked by laser cut pairs of corresponding male and female components. The contours of the male and female components generally preclude the respective male components received therein from escaping from the female components even as the dynamics of the vascular or other system within which the stent is placed urges a male component on an opposite side of a set out of a corresponding female component. Rotational movement of the mechanically interlocked male components remains generally unimpeded. Designated pairs of mechanically interlocked strut sections within a set of longitudinally adjacent strut sections are diametrically opposed, or otherwise oriented, relative to one another which keeps the sections together. Sets of neighboring mechanically interlocked adjacent strut sections are arranged out of phase relative to one another in order to increase the rigidity and flexibility of the stent.

### BACKGROUND OF THE INVENTION

1. Field of the Invention

The invention generally relates to a stent having mechanically interlocking struts and methods for making the same. More specifically, the invention relates to stents having mechanical interlocks comprised of male and female components that are movably connected to one another at ends of designated sets of adjacent struts.

2. Related Art

Intraluminal endovascular stents are well-known. Such stents are often used for repairing blood vessels narrowed or occluded by disease, for example, or for use within other body passageways or ducts. Ideally, the stent will conform to the contours and functions of the blood vessel or other body passageway in which the stent is to be deployed. Increased flexibility of the stent generally eases delivery of the stent and increases the conformability of the stent to the environment in which the stent is deployed.

FIGS. 1a & 1b show structures that have been used as intraluminal vascular stents in the past. Such prior art stents 1 and 2, respectively, have included a cylindrical body comprised of metal elements joined to one another in a manner that permits flexing of the cylindrical body along its longitudinal axis, as disclosed in U.S. Pat. Nos. 4,733,665 and 4,776,337.

FIG. 2 shows other stents 3 that have been used in the past. Such other stents 3 have been comprised of a cylindrical body of metal elements with spiral loops to join the metal elements to one another, as disclosed in U.S. Pat. Nos. 6,238,409 and 6,565,600, both of common assignment herewith. The spiral loops contribute to the flexibility of the stent about its longitudinal axis.

FIG. 3 shows still other known stents 4 comprised of a cylindrical body of metal elements in a generally repetitive scheme of looped metal struts joined by flexible members. The flexible members are oriented generally tranverse relative to the metal struts and are connected to ends of offset metal struts, as disclosed in U.S. Pat. No. 6,790,227 also of common assignment herewith. Varying the length or shape of the struts or the flexible members, or changing the manner in which the flexible members are connected to ends of offset struts contributes to the stent's flexibility.

Still further, FIG. 4 shows other known stents 5 that provide flexible members connecting the end of one strut to the end of another strut along with an additional interlocking element that connects sides of adjacent struts to one another, as in U.S. Pat. No. 6,562,067 also of common assignment herewith.

Although the above described stents can be effective in terms of stenting open an occluded or otherwise blocked vessel or passageway, the construction of such stents still pose flexibility limitations that can render delivery of the stents difficult or unreliable and that can hinder the stent's conformability to the bodily dynamics of the environment in which it is deployed. Where delivery difficulties occur, as where the stent's rigidity and flexibility do not readily negotiate the tortuous vessel or other passageway to be traversed to locate the stent as desired, portions of the stent may contact and damage the interior lining of previously healthy vessels. Undesirable emboli may occur within the vessel as a result. Further, even after successful delivery of the stent to a desired location, the non-conformability of less flexible stents can result in rupture or other damage to the vessel or passageway in which the stent is located.

In view of the above, a need exists for systems and methods that improve the flexibility and rigidity of stents in order to render delivery of the stent to a vessel or other passageway easier and more reliable. A further need exists for systems and methods that improve the flexibility of stents and render the stent more readily compliant with the vessel or passageway within which the stent is located once delivery is effected.

### SUMMARY OF THE INVENTION

The systems and methods of the invention provide a stent with increased flexibility. The increased flexibility of the stent more easily accommodates delivery of the stent to a blood vessel or other passageway in the body of a patient. The increased flexibility of the stent also more readily conforms the stent to the bodily dynamics of the blood vessel or passageway in which the stent is eventually located. The systems and methods of the invention further provide sufficient rigidity to the stent to reliably deliver the stent to its intended location in the body.

According to the systems and methods of the invention, a cylindrically-shaped stent having a longitudinal axis is provided. The stent is comprised of a first end, a second end and an intermediate section therebetween. The longitudinal axis thus extends within the cylindrical stent from the first end to the second end of the stent. The intermediate section is further comprised of a series of adjacent strut sections that are aligned substantially parallel relative to the longitudinal axis of the stent so as to substantially form the cylindrical shape of the stent. Each strut section is comprised of an undulating wave. A closed end of the undulating wave of each strut section is generally aligned with a closed end of the undulating wave of a longitudinally adjacent strut section such that the closed ends of designated pairs of longitudinally adjacent strut sections oppose one another. A mechanical interlock joins the closed ends of the designated pairs of opposed longitudinally adjacent strut sections. Two or more pairs of mechanically interlocked closed ends of longitudinally adjacent strut sections comprise a set, wherein the mechanically interlocked pairs within a set are diametrically opposed, or otherwise oriented, relative to one another within the set. Neighboring sets of the mechanically interlocked strut sections are oriented out of phase relative to one another. The more neighboring sets of mechanically interlocked strut sections that are connected to one another, the longer the cylindrical stent becomes.

According to the systems and methods of the invention, each mechanical interlock is comprised of a male component extending from the closed end of one strut section, and a female component extending from the closed end of a correspondingly aligned adjacent strut section. Each male component is thus received within a corresponding female component.

According to the systems and methods of the invention, the male (ball) component of the mechanical interlock is restricted from being pushed too far inwardly toward the center of the device (stent) through the female component as an interference with the female component exists due to a wedge-shaped cross sectional geometry of both the male and female components. Likewise, the female component of the mechanical interlock is unable to be pushed outwardly beyond the male component because of this interference. Wedge shaped cross-sectional geometry is inherent to all longitudinal (parallel to the tube axis) stent geometry cut with traditional laser techniques. This invention leverages the wedge shaped cross-sectional geometry and the manner in which a laser system cuts tubing in order to create a flexible ball and socket mechanical interlock that remains together and needs no post assembly.

Traditional tube cutting laser systems incorporate a fixed laser beam and use linear and rotational stages to manipulate tubing under this beam in order to cut complex geometry in the form of a stent. During this process, the laser beam is stationary above the center axis of the tube and the tube moves longitudinally under the beam while rotating, such that the laser beam is always directed toward the center axis of the tubing. It is this method of cutting that creates the wedge shaped cross-sectional geometry that runs the length of the stent, as seen in FIG. 11 and the interlock cross-section in FIG. 12.

As previously mentioned, according to one embodiment a set is comprised of two or more pairs of mechanical interlocks that are diametrically opposed. This diametrically opposed relationship is what holds the pairs of mechanical interlocks together. Considering that each male component of a pair is attached to each other through an undulating strut section and each female component is attached in the same fashion, any movement to either a male or female component will result in the same movement by the other component in the pair. Therefore, considering just the relationship of the male to the female component, the male has limited movement inward yet can be pushed outward; however, examining the entire system, outward movement of the male component would cause inward movement of the diametrically opposed male component which is not possible or is limited due to the wedge interference (see FIG. 13 which is a cross-sectional view through an interlock pair). Likewise, in relation to just the male component, the female component has very limited movement outward but can move inward, however, the diametrically opposed relationship prohibits such movement. This concept is what keeps the mechanical interlocks together while allowing the components to rotate freely.

In a preferred embodiment, the series of struts, and then the male and female components extending from closed ends thereof, are laser cut from a tubular stent material of a known thickness. The included angular dimensions (see FIG. 7c) of the male and female components are achieved by the simultaneous laser cutting of the male and female components from the same tubular stent material. The male component is thus freely rotatable within the female component after cutting, and is slightly inwardly movable within the female component in which it is retained according to the degree of interference provided by the angular dimensions of the male and female components.

The degree of interference between the male and female components is directly proportional to the included angle such that an increase in the angle leads to a greater degree or percentage of interference. In other words, the smaller the included angular dimension of the male and female components, the greater the inward movement of the male component within the corresponding female component. When considering the standard method of laser cutting discussed above, the included angle is directly related to the tubing outside diameter (OD) and the male component width. The relationship is as follows: an increase in male component width increases the included angle and vice versa, and an increase in tubing OD would decrease the included angle and vice versa.

Two other attributes of the device and/or method that affect the degree of interference between the male and female components are the tubing/device wall thickness and the kerf width (width of material removed by the laser beam). For example, the degree of interference between the male and female components will increase with an increase in wall thickness and decrease with a decrease in wall thickness. Furthermore, the degree of interference will decrease as the kerf width increases and increase as kerf width decreases.

According to the systems and methods of the invention, the greater the degree of interference between the male and female components, the greater likelihood the mechanical joints will remain together and not separate when external forces are applied. Increasing the included angle and/or increasing the thickness and/or decreasing the kerf width can increase the degree of interference. Changes to any or all of these attributes may be necessary to retain the male component within the female component. Furthermore, changes to these attributes may be needed to compensate for the material lost during electropolishing if the device is to be electropolished. Electropolishing the device may lower the degree of interference considering the width between the male and female component (kerf width) may widen.

Ideally, the neighboring sets of diametrically opposed pairs of adjacent strut sections are 90° out of phase relative to one another. In this manner, the stent is provided with sufficient rigidity for delivering the stent to an intended location, and with sufficient flexibility to more readily conform to the changing dynamics of the system within which the stent is located. The diametrically opposed relationship of each set of mechanically interlocked pairs of adjacent strut sections helps maintain the mechanically interlocked relationship of the male component ball within the female component even when the ball is urged outward by pressures applied to the stent: Of course, orientations of neighboring sets other than 90° out of phase are possible too according to the systems and methods of the invention.

Another embodiment of the systems and methods of the invention, involves manufacturing individual stent sections comprised of struts and one portion, male or female, of the mechanical interlock on both sides of said section as seen in FIG. 14. Thereafter, the designated pairs of male and female components comprising a set are assembled to form the mechanically interlocked stent. Preferably, shape memory materials comprise the various components of the stent according to this embodiment, although other materials may be used. As in earlier embodiments, the more neighboring sets of mechanically interlocked pairs of longitudinally adjacent strut sections that are assembled, the longer the stent becomes. As also in the preferred embodiment, the included angular dimensions of the male components and of the female components, in addition to the thickness of the material from which the male and female components are cut, determine the amount of interference between the male component and the corresponding female component within which it is retained, while permitting free rotational movement of the male component within the female component, thus enhancing the flexibility of the stent overall. The male and female components of each set are thus maintained in an interlocked relationship even in the presence of changing bodily dynamics.

According to the systems and methods of the invention, the stent is comprised of biocompatible materials known or later developed in the art, and the male and female components of the mechanical interlock are laser cut from the same material as comprises the stent.

In another embodiment of the systems and methods of the invention, a conventional laser system like the one previously discussed is not used to cut the device. Instead, the laser beam is not fixed and is mounted to an X-Z stage so it can move transversely (x-stage) across the outside of the tubing and vertically (z-stage) to keep the laser beam focused. These additional degrees of freedom allow the designer to increase the included angle of the male and female components without increasing the overall size of these components (see FIG. 15). The benefits of increasing the included angle are mentioned above.

The above and other features of the invention, including various novel details of construction and combinations of parts, will now be more particularly described with reference to the accompanying drawings and claims. It will be understood that the various exemplary embodiments of the invention described herein are shown by way of illustration only and not as a limitation thereof. The principles and features of this invention may be employed in various alternative embodiments without departing from the scope of the invention.

### BRIEF DESCRIPTION OF THE DRAWINGS

These and other features, aspects, and advantages of the apparatus and methods of the present invention will become better understood with regard to the following description, appended claims, and accompanying drawings where:

FIGS. 1a and 1b illustrate prior art stents.

FIG. 2 illustrates another prior art stent.

FIG. 3 illustrates another prior art stent.

FIG. 4 illustrates another prior art stent.

FIGS. 5 illustrates a perspective view of a mechanically interlocked stent according to the invention.

FIG. 6 illustrates a perspective view of male and female components of a mechanically interlocked flex connector according to the invention.

FIG. 7a illustrates a schematic view of the mechanically interlocked stent of FIG. 6 with a cross-sectional line A-A bisecting the male and female components of the mechanical interlock according to the invention.

FIG. 7b illustrates a cross-sectional view of the male and female components taken from the line A-A of FIG. 7a according to the invention.

FIGS. 7c and 7d illustrate various dimensional aspects of the male and female components of a mechanical interlock.

FIGS. 7e and 7f illustrates various dimensional aspects of the male and female components of a mechanical interlock.

FIG. 8 illustrates a cross-sectional view of another arrangement of mechanically interlocked strut sections according to the invention.

FIG. 9 illustrates an apparatus and method for laser cutting a mechanically interlocked stent on-center according to various methods of the invention.

FIG. 10 illustrates another apparatus and method for laser cutting a mechanically interlocked stent with a laser system that can be programmed for off-center cutting, to increase the included angle of the male and female components without changing any other attributes.

FIG. 11 illustrates a laser cutting tubing resulting in wedge-shaped cross-sectional geometry of male and female components according to the invention.

FIG. 12 shows an isometric view of a mechanically interlocked stent that has been cross-sectioned through a set of mechanical interlocks to help visualize the wedge-shaped cross-sectional geometry.

FIG. 13 shows an isomeric view of a mechanically interlocked stent that has been cross-sectioned through a set of mechanical interlocks as an external force is applied to it.

FIG. 14 is an isometric view of two separate stent sections comprised of the undulating strut section and the corresponding male and female components.

FIGS. 15a and 15 illustrate schematically how off-center laser cutting will increase the included angle of the male and corresponding female components, making a more pronounced wedge.

FIG. 16 shows another embodiment in which the shaft of the male component is attached to the valley of the radial arc of the undulating strut section.

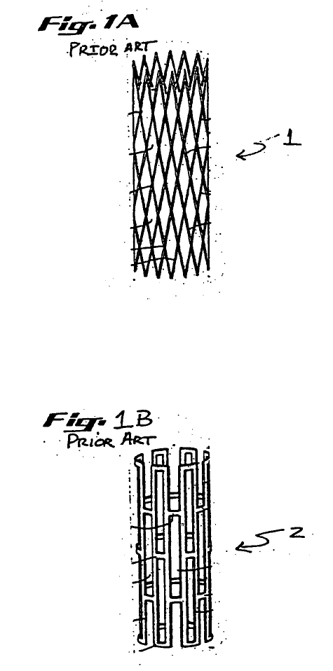

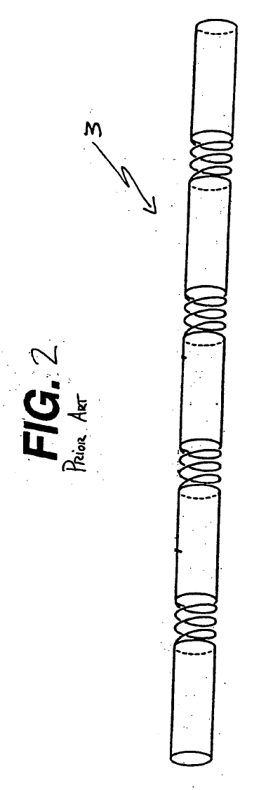

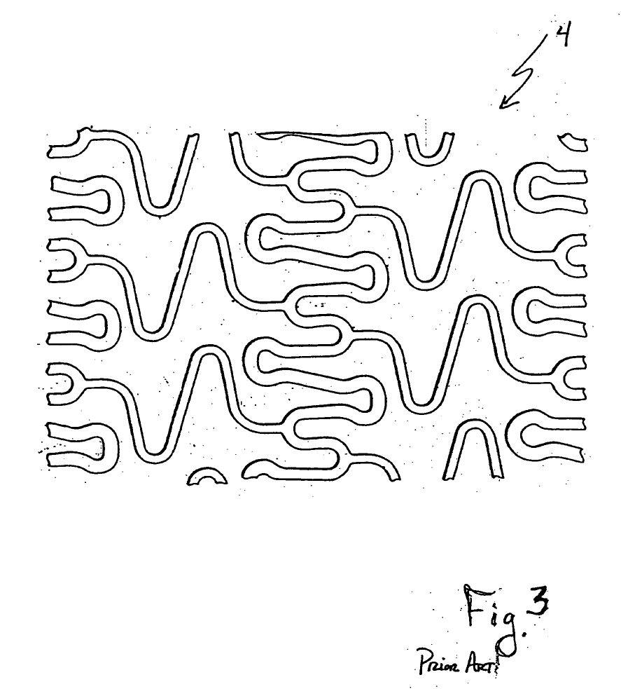

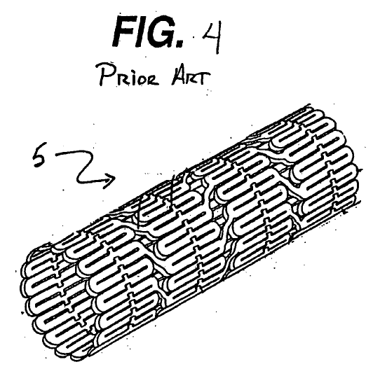

### DETAILED DESCRIPTION OF THE INVENTION

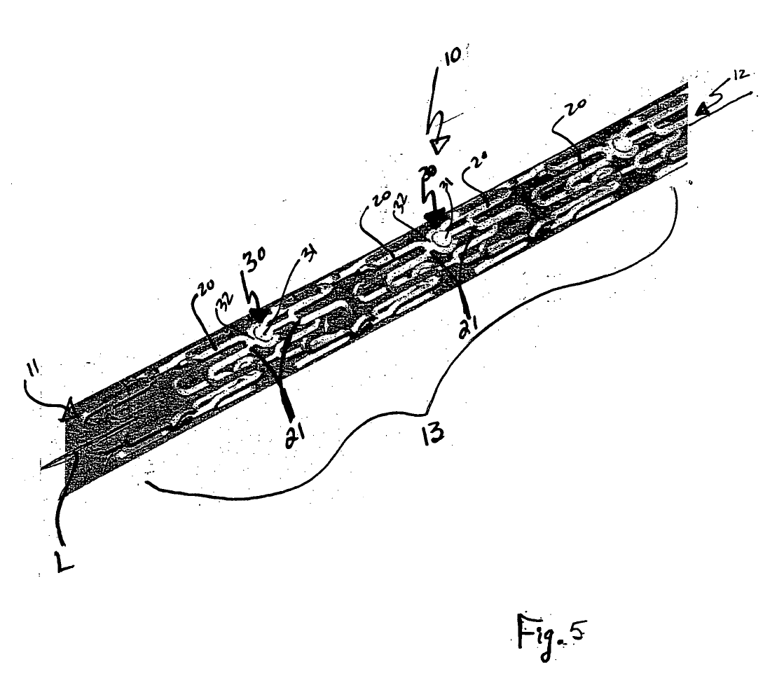

FIG. 5 illustrates a cylindrical stent 10 having a first end 11, a second end 12 opposite the first end 11, and an intermediate section 13 extending therebetween the first and second ends 11, 12. A longitudinal axis L extends throughout the stent 10 between the first and second ends 11, 12 as well. Hereafter, any reference to “inward” or “inwardly” movement is understood as movement towards the longitudinal axis L, unless otherwise indicated. The intermediate section 13 of the stent 10 further comprises a series of adjacent strut sections 20 comprised of undulating waves that extend longitudinally between the first and second ends 11, 12. A closed end 21 of the undulating wave of one strut section 20 is generally aligned opposite a closed end 21 of the undulating wave of an adjacent strut section 20 so as to form designated pairs of longitudinally adjacent strut sections 20. Each designated pair of longitudinally adjacent strut sections 20 are connected via a mechanical interlock 30. The series of longitudinally adjacent strut sections 20, including those that are mechanically interlocked, thus extend in substantially parallel relationship relative to the longitudinal axis L of the stent to substantially form a circumference of the cylindrical stent 10. Two or more of the mechanically interlocked pairs of longitudinally adjacent strut sections 20 comprise a set 25, wherein the pairs within a set 25 may be diametrically opposed, or otherwise oriented, relative to one another. Neighboring sets 25 are oriented generally out of phase relative to one another. The more neighboring sets 25 of mechanically interlocked strut sections 20 that are connected to one another via the mechanical interlock 30, the longer the cylindrical stent 10 becomes.

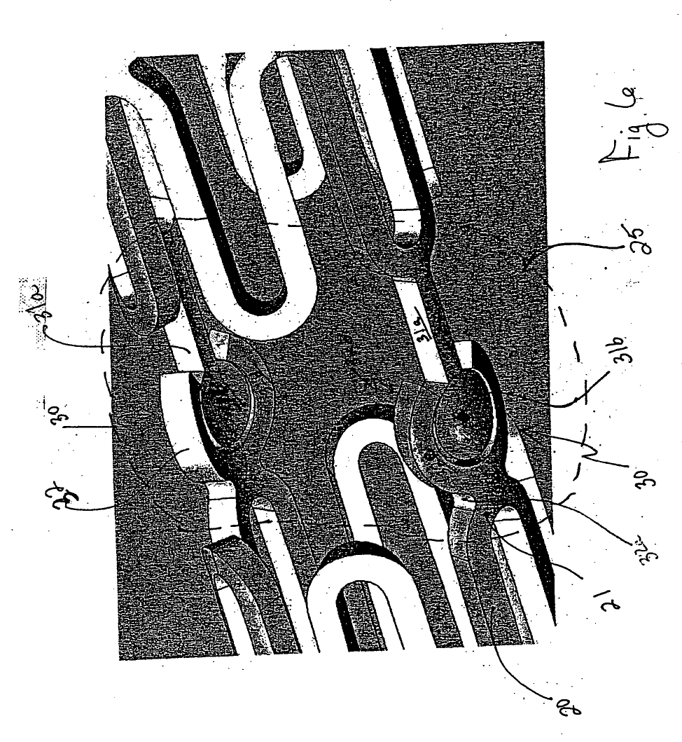

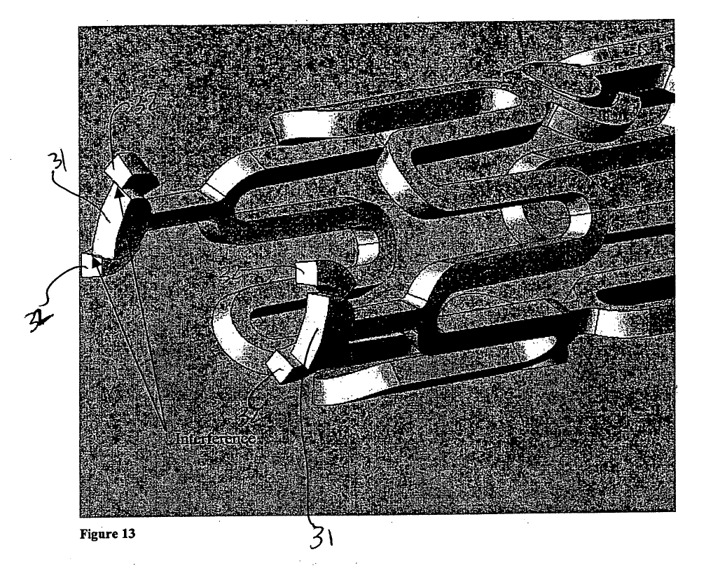

Each mechanical interlock 30 is comprised of a male component 31 having a shaft portion 31a, a ball 31b and side surfaces 31c (FIG. 6) and a female component 32 having a shaft portion 32a, a receptacle 32b and side surfaces 32c (FIG. 6). In some embodiments, the male and female components are formed simultaneously so that no further assembly of the male component with the female component is required to comprise the stent with the mechanical interlock 30, whereas in other embodiments the male and female components 31, 32 with the attached strut sections (FIG. 14) are separately formed requiring assembly thereof to comprise the mechanically interlocked stent 10. In either case, the male and female components 31, 32 within a set 25 join the designated pairs of otherwise closed ends 21 of the longitudinally adjacent strut sections 20 (set 25 circled in dashed lines in FIG. 6), shown as diametrically opposed pairs, i.e., 180° apart, of adjacent strut sections 20 in FIG. 6.

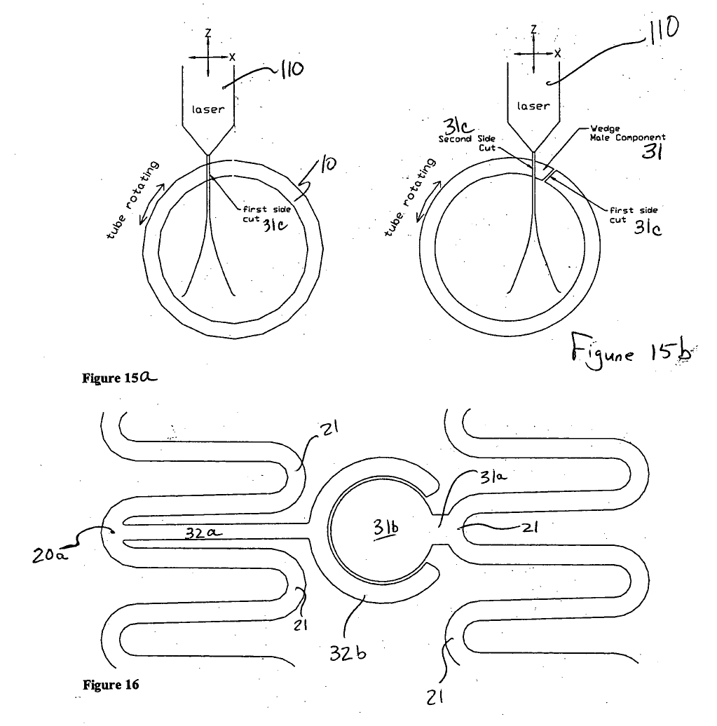

Referring still to FIG. 5, each set 25 of mechanically interlocked strut sections 20 is out of phase with its neighboring sets 25 of mechanically interlocked strut sections 20. The sets 25 may be 90° degrees out of phase with one another, as shown in FIG. 5, or may be other than 90° out of phase with one another. The male component 31 of the mechanical interlock 30 is provided on the closed end 21 of one strut section 20, whereas the female component 32 of the mechanical interlock 30 is provided on the closed end 21 of a corresponding longitudinally adjacent strut section 20. However, in some embodiments the male or female component is attached to an interior radius of the closed strut section 20, also referred to as the radial arc 20a, and its mating partner is attached to the apex of the closed end 21 of a corresponding longitudinally adjacent strut section 20, as can be seen in FIG. 16. For this embodiment, a closed end of each undulation strut section does not align with a closed end of an adjacent strut section.

The amplitude of the undulating waves of the strut sections 20 shown in FIG. 5 are generally constant, although the artisan should appreciate that strut sections comprised of undulating waves of varying amplitudes may also be used to comprise the strut sections according to the invention. In the preferred embodiment, the closed ends 21 of the longitudinally adjacent strut sections are aligned with one another to permit the mechanical interlock 30 of sets 25 of designated pairs of adjacent strut sections, as discussed herein according to the invention. In another embodiment the strut sections are also aligned, however there is no phase shift in the undulations such that peak of one strut section aligns with the valley of the adjacent strut section as seen in FIG. 16. Where varying amplitudes are used, a shaft 31a of the male component 31, or a shaft 32a of the female component 32 may be altered by lengthening or shortening the components appropriately to accommodate the differing amplitudes, as should be readily appreciated by the artisan.

As shown in FIGS. 5 & 6, each set 25 of mechanically interlocked pairs of adjacent strut sections 20 is staggered, i.e., 90° out of phase, with its neighboring sets 25 of mechanically interlocked pairs of adjacent strut sections 20. Staggering the mechanical interlock 30 of neighboring sets 25 of opposed pairs of adjacent strut sections 20 in this or similar manner increases the rigidity of the stent 10, which assists delivery of the stent 10, while still providing improved flexibility of the stent 10. Although the staggering of neighboring sets 25 of mechanically interlocked pairs of adjacent strut sections 20 is ideally 90° out of phase with neighboring sets 25 of mechanically interlocked pairs of adjacent strut sections 20, as shown in FIG. 5, the artisan should readily appreciate that staggering the neighboring sets of mechanically interlocked pairs of adjacent strut sections can also be other than at 90°.

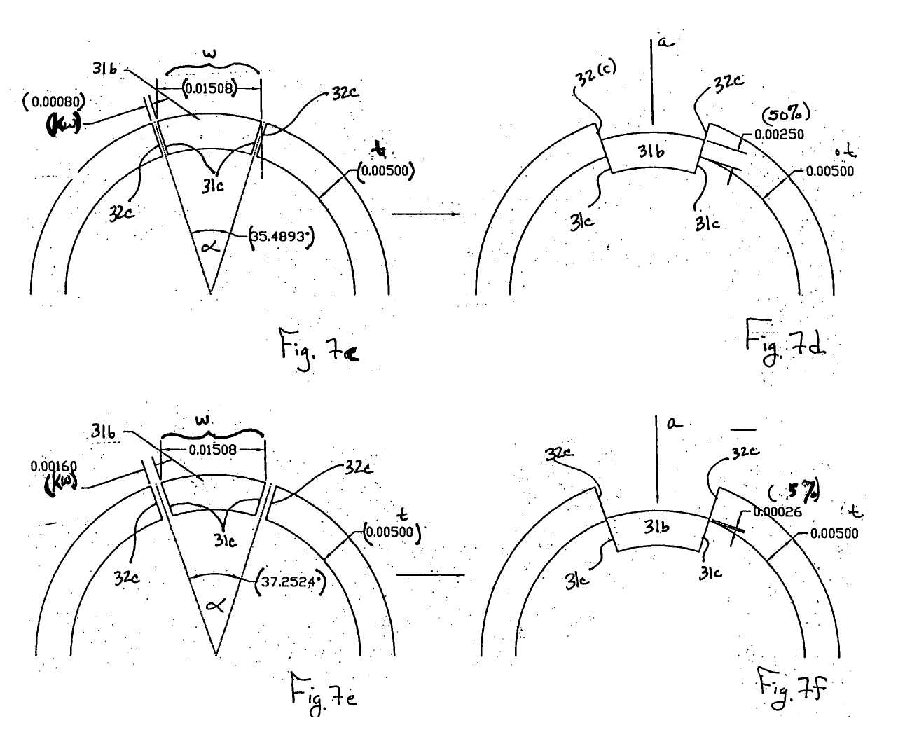

For example, as shown in FIG. 8, more than two mechanical interlocks 30 may be provided within a set 25 of paired adjacent strut sections 20 such that the mechanical interlocks 30 are other than diametrically opposed from one another (FIG. 8). Where three mechanical interlocks 30 are used, for example, the mechanical interlocks may be 120° apart from one another as they join respective pairs of adjacent strut sections 20. Moreover, the neighboring set 25 of mechanical interlocks 30 may be 60° out of phase with one another instead of 90° out of phase with one another as described above with respect to FIG. 5. Of course, other configurations are also contemplated according to the invention, as should be readily appreciated by the artisan.

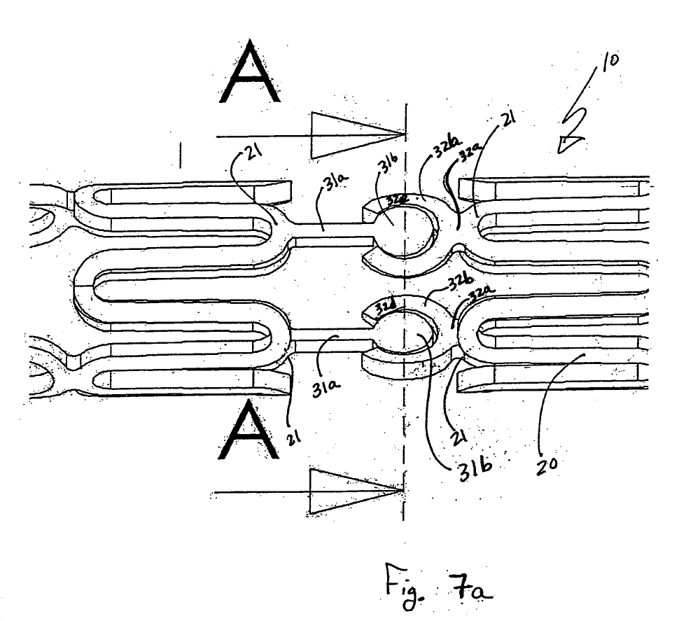

FIG. 6 shows in more detail the mechanical interlock 30 according to the invention. The mechanical interlock 30 connects the set 25 (dashed line) of pairs of longitudinally adjacent strut sections 20 to one another as discussed above. Each mechanical interlock 30 is comprised of the male component 31 and the female component 32. The male component 31 includes a shaft portion 31a and a ball 31b. The female component 32 includes a shaft 32a (that is abbreviated in FIG. 6), and a semi-circular receptacle 32b. Angled side surfaces 32c (shown best in the inset of FIG. 7b) of the female component 32 correspond to angled side surfaces 31c of the male component. When received within the receptacle 32b of the female component 32 therefore, the ball 31b of the male component 31 is thus rotatable and slightly inwardly movable, i.e., movable towards the longitudinal axis L, while still being retained within the female component.

Where the male and female components 31, 32, respectively, are formed simultaneously as by laser cutting, discussed in more detail below, the angled side surfaces 31c and 32c of the male and female components 31 and 32, respectively, are automatically synchronized such that male component 31 is received within the female component 32 without requiring further assembly of the stent 10. Where the male and female components 31 and 32 are separately formed, also discussed further below, then omission of the kerf width (material removed by laser beam) that otherwise occurs in the simultaneous formation method, permits an even tighter, or more precise, fit of the male component 31 within the female component 32, as the male component 31 can be made slightly larger: Thereafter, each male component 31 is appropriately positioned and retained within a corresponding female component 32 to comprise the mechanically interlocked stent while permitting rotational movement of the male component 31 within the respective female component 32 in which it is retained.

Referring still to FIG. 6, the angled side surfaces 32c of the female components 32 helps to retain the ball 31b of the male components 31 by colliding or interfering with a portion of the angled side surfaces 31c of the male component 31 as the male component moves inwardly towards the longitudinal axis L of the stent due to the changing dynamics of the vascular or other system in which the stent 10 is emplaced. In this regard, in each set 25 of mechanically interlocked strut sections 20, the angled side surfaces 32c of the female component 32 in which the ball 31b of a corresponding male component 31 is received generally precludes the ball 31b of the male component 31 in the female component 32 on one side of the stent from unhinging or escaping from the female component 32 by moving too far inwardly, i.e., towards the longitudinal axis L, even as dynamics urge the ball 31b in the female component 32 on the opposite side of the stent outwards, i.e., towards the vessel and away from the longitudinal axis L. At the same time, rotational movement of the male component 31 in the female components 32 on all sides of the stent 10 is generally unrestricted. In this manner, enhanced flexibility of the stent is achieved. The angled side surfaces 31c and 32c of the male and female components 31 and 32, respectively, can thus be cut with different angles in order to accommodate or limit different amounts of inward movement of the balls 31b of the corresponding male components 31 as desired. To this end, other parameters, such as kerf width (kw), stent wall thickness (t), or other dimensions of the male or female components (FIGS. 7a-7f) may also be altered such that a range of inward movement of the male component may be achieved or limited, as desired.

The correspondingly angled side surfaces 31c, 32c of the male and female components 31, 32 thus help to maintain the alignment of the stent 10 by maintaining at least one of the male components 31 in place within a female component 32 of the mechanical interlock 30 in each set 25 of mechanically interlocked longitudinally adjacent strut sections 20 according to the invention. The ability of the stent 10 to conform to changing dynamics within the vascular or other system within which the stent is emplaced is thus enhanced by the increased flexibility provided by the rotational movements of some male components 31 within their corresponding female components 32 while maintaining the mechanical interlocking of adjacent strut sections 20 of each set 25 as needed.

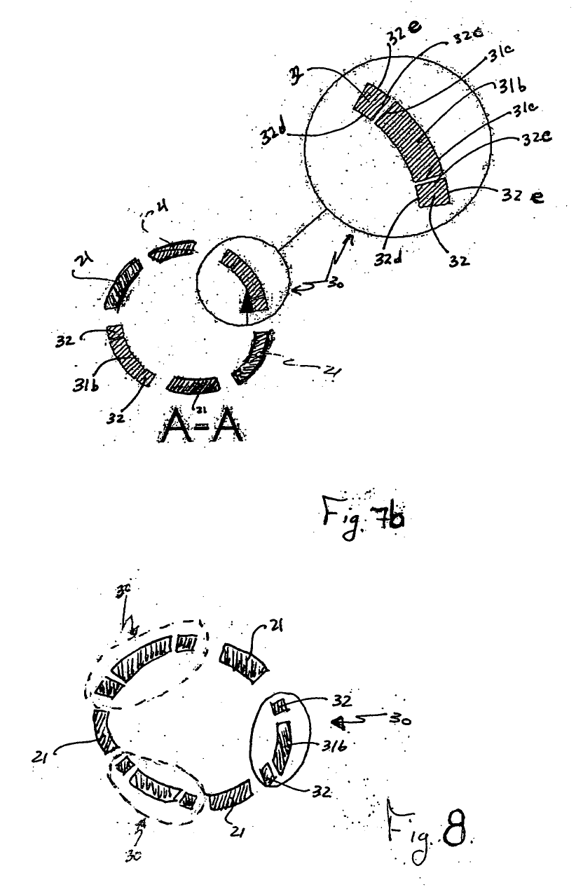

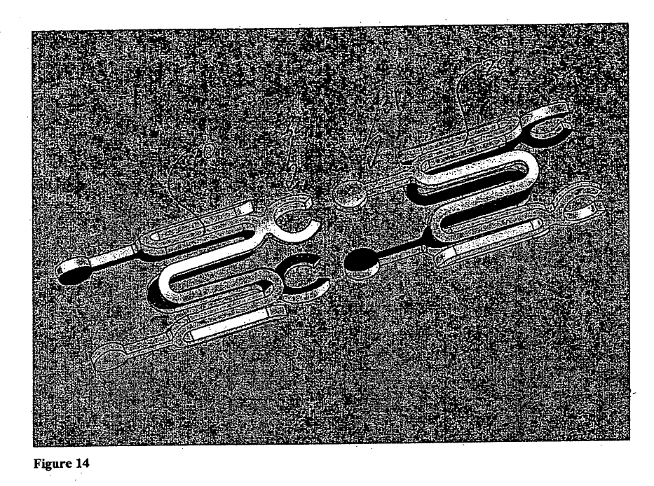

FIG. 7a illustrates schematically the stent 10 of FIG. 6, wherein a cross-sectional line A-A bisects the ball 31b of the male component 31 that is received within a corresponding one of the female components 32. FIG. 7b illustrates the cross-sectional view of the ball 31b received in the female component 32 taken from the line A-A of FIG. 7a. The circled inset in FIG. 7b shows in more detail the configuration of the ball 31b of the male component as it is seated within the corresponding female component 32. Ideally, the stent 10, including the strut sections 20, and the male component 31 and female component 32, is formed by laser cutting as discussed in greater detail further below. The laser cutting can be conventional on-center laser cutting methods, one of which is detailed herein with respect to FIG. 9, or the laser cutting can be off-center or rotational laser cutting methods permitting increased angular dimensions of the various components, for example, as in FIGS. 15a & 15b.

Referring still to FIGS. 7a & 7b, for example, with respect to the male and female components 31, 32 in particular, the shaft 31a, ball 31b and angled side surfaces 31c of the male component 31, and the shaft 32a, receptacle 32b and angled side surfaces 32c of the female component 32 are laser cut from the same tube as comprises the stent 10. The male and female components 31, 32 may be simultaneously laser cut as described in greater detail further below with respect to FIG. 9, or they may be separately laser cut and then assembled to comprise the mechanically interlocked stent 10, as also described further below.

Laser cutting the male and female components 31, 32 simultaneously results in the male and female components 31, 32 being automatically assembled, wherein the side surfaces 31c of the male component 31 and the side surfaces 32c of the female component 32 synchronously fit one another, as evident in the inset of FIG. 7b. Cutting the angled side surfaces 31c and 32c of the male and female components 31 and 32 in this manner also readily accommodates the rotational movement of a corresponding ball 31b of a male component 31 retained within a female component 32. By changing the included angle of the side surfaces 31c and 32c of the male and female components 31, 32, or by changing the thickness of material comprising the stent wall, for example, the amount of interference between that of the male component 31 and the female component 32 can be controlled or limited, while permitting unrestricted rotational movement of the male component 31 within a corresponding female component 32, which in turn enhances the flexibility of the stent 10 overall. Of course, altering the the kerf width (width of material removed by the laser) will also alter the amount of interference between the male component 31 and a female component 32. Furthermore, the included angle between side surfaces 31c and 32c of the male and female components 31, 32 is directly related to the width of the male component 31 and the outside diameter of the stent and can be changed by adjusting these attributes.

FIGS. 7c and 7d illustrate various dimensions for a male component 31 and female component 32 cut simultaneously using a laser beam. The male component 31 and female component 32 are cut from the same tubular material from which the struts 20 of the stent 10 are cut. The struts 20 are generally cut first, and then the outside of the male and female components 31, 32 are cut, and finally the material between the male and female components 31, 32 is cut with one pass of the laser beam. Thus, once the male and female components 31, 32 are cut and the material between them is cut, the stent 10 is formed with no further assembly required.

Referring still to FIGS. 7c and 7d and also to 7e and 7f, these figures demonstrate how changing different method attributes affect the mechanical interlock, in this case the affect of changing the kerf width. FIGS. 7c and 7d represent a stent fabricated with a laser that produces a kerf width equal to 0.0008″ while FIGS. 7e and 7f represent a stent fabricated with a laser that produces a kerf width equal to 0.0016″. FIG. 7c and 7e represent the stent in equilibrium while FIG. 7d and FIG. 7f represents the stent with a possible external force. All other attributes (male component size, tubing outside diameter, material thickness), except for the included angle which is affected by this kerf change, are exactly the same, however the amount of interference between male and female components differ. In fact, FIG. 7d shows that if an external force is applied to the male component of this configuration an interference will occur at 50% into the material thickness while FIG. 7f reveals an interference of 5% because of the greater kerf width. Of course, as the artisan should readily appreciate, modifying either or all of the stent material thickness (t), kerf width, the included angle α, or other dimensions may modify the amount of interference between the male ball 31b and the female component 32 from 0 to 100%, as desired.

FIG. 8 illustrates, in cross-section, another arrangement of the male and female components 31, 32 as if taken along the line A-A of a stent comparable to that of FIG. 7a. As shown in FIG. 8, however, the closed ends 21 of each set 25 of paired adjacent strut sections are connected via mechanical interlocks 30 provided at positions other than diametrically opposed to one another. In this regard, more than two sets of male and female components 31, 32 connect each set 25 of adjacent strut sections according to the invention. The mechanically interlocked sets 25 of adjacent strut sections 20 in the arrangement shown in FIG. 8 would thus be other than 90° out of phase, for example 60°, with neighboring sets thereof as discussed above. Of course, the artisan will readily appreciate that such sets can be arranged out of phase by other amounts of degree relative to one another as desired.

In practice, any of several conventional laser cutting methods may be used to fabricate the mechanically interlocked stent 10 according to the systems and methods of the invention. Thus, without limiting fabrication of the mechanically interlocked stent according to the invention to methods described herein, one method of making the cylindrical stent having sets of mechanically interlocked adjacent strut sections according to the systems and methods of the invention comprises providing a cylindrical tube of bio-compatible material 40 as shown in FIG. 9. The bio-compatible material may be any known or later developed material appropriate for use as a stent deployed within anatomical systems according to the invention.

The materials for the stent should exhibit excellent corrosion resistance and biocompatibility. In addition, the material comprising the stent should be sufficiently radiopaque and create minimal artifacts during MRI.

The most widely used material for stents is stainless steel, particularly 316L stainless steel. This material is particularly corrosion resistant with a low carbon content and additions of molybdenum and niobium. Fully annealed, stainless steel is easily deformable. Alternative materials for stents include tantalum, platinum alloys, niobium alloys, and cobalt alloys. In addition, other materials such as polymers and bioabsorbable polymers may be used for the stents made according to the systems and methods described herein.

Where the male and female components are separately laser cut, a suitable material is shape memory material such as nitinol, a nickel-titanium alloy or other material having similar elasticity.

Of course, the disclosure of various materials comprising the stent should not be construed as limiting the scope of the invention. One of ordinary skill in the art would understand that other material possessing similar characteristics may also be used to comprise the stent according to the systems and methods described herein.

The stent may be fabricated using several different methods as described herein, for example. Typically, the stent is made from tubing material. The various methods disclosed herein also should not be construed as limiting the scope of the invention. One of ordinary skill in the art would understand that other methods may be employed to form the stent and its components including the mechanical interlock as described herein.

The method shown in FIG. 9 further comprises providing an apparatus 50 to which the tube 40 is mounted during cutting. The apparatus 50 generally comprises an X-Y table 51, a rotary support 52, a focusing device 53, a laser beam 54 and a computer 60. The X-Y table 51 moves generally horizontally in the X direction according to the view shown in FIG. 9, whereas the rotary support 52, which is fixed to the X-Y table 51, rotates the tube 40 about its longitudinal axis L. The focusing device 53 and laser beam 54 are generally fixed relative to the X-Y table 51 and rotary support 52 such that the tube 40 moves in order to achieve the desired laser cuts. To this end, the focusing device 53 and laser beam 54 may be housed in a housing 55 in fixed relation to the X-Y table 51 and rotary support 52 once cutting begins.

The focusing device 53 focuses the laser beam 54 to locations on the tube 40 that are to be cut. The focusing device may comprise a lens, a mirror or other suitable focusing means. Preferably, the laser beam 54 is focused in small enough regions during laser beam cutting that energy from the laser beam 54 does not penetrate or otherwise negatively impact surfaces of the tube 40 that are not intended to be cut. To this end, liquids (mostly water) may be provided to the center of the tube 40 through a hose 56 connected to an end of the rotary support 52, for example, while laser cutting occurs in order to attenuate the laser beam energy. Of course, the artisan will appreciate that liquids other than, or in addition to, water may be provided to attenuate the laser beam energy according to the invention. Likewise, the liquids may be provided to the tube 40 from locations other than as described herein in order to attenuate laser beam energy so as not to detrimentally affect unintended surfaces of the tube 40. Of course, the artisan will further appreciate that where the laser beam is sufficiently exact as to minimize impact on unintended surfaces of the tube 40 during cutting thereof, attenuating liquids may be omitted.

Referring still to FIG. 9, the computer 60 is connected and programmed to cause the X-Y table 51 and the rotary support 52 to move at appropriate speeds and directions such that the laser beam 54 executes a multiplicity of cuts in the tube 40 to define the various struts 20, and the male and female components 31, 32 according to the invention. To this end, the computer 60 is programmed to move the X-Y table 51 lengthwise in the X direction while the rotary support 52 is simultaneously programmed to rotate the tube mounted thereof about the longitudinal axis L of the tube 40. During this combination of motions, the laser beam 54 is focused through focusing means 53 to laser cut one set of longitudinally adjacent strut sections 20 comprised of undulating waves having closed ends 21 as described hereinabove. The strut sections 20 thus are cut to include the undulating waves having closed ends 21 as described above.

Thereafter, the X-Y table 51 and rotary support 52 bearing the tube 40 are positioned to start cutting the male components 31 and the female components 32 from designated pairs of closed ends 21 of the strut sections 20 that have already been cut. Preferably, the outsides of the male and female components 31, 32 are cut and then the area between the male and female component 31, 32 is cut with one pass of the laser beam at which point sides 31c and 32c are created. The mechanical interlock 30 of the stent is thus achieved according to the systems and methods of the invention. FIG. 5 illustrates generally a laser cut stent 10 made using the methods described herein.

More specifically, during laser cutting of the male components 31 the shaft 31a, the ball 31b and the side surface 31c of the male components 31 are formed. Similarly, during laser cutting of the female components 32 as described above, the abbreviated shaft 32a, semi-circular receptacle 32b and angled side surfaces 32c of the female components 32 are formed. By varying the included angle of the side surfaces 31c, 32c, through changing the tubing outside diameter (OD) and/or the male component width or varying the kef width or material thickness (t), the amount of interference between the male component 31 and the female component 32 is controlled, while permitting unrestricted rotational movement of the male component in the female component. Further, because the various strut sections 20 and the male and female components 31, 32, respectively, of the mechanical interlock 30 are fabricated from the same tube by the laser cutting technique described herein, wherein the angled sides 31c, 32c of male and female components 31, 32 are cut simultaneously, no additional assembly of the mechanical interlock 30 is required.

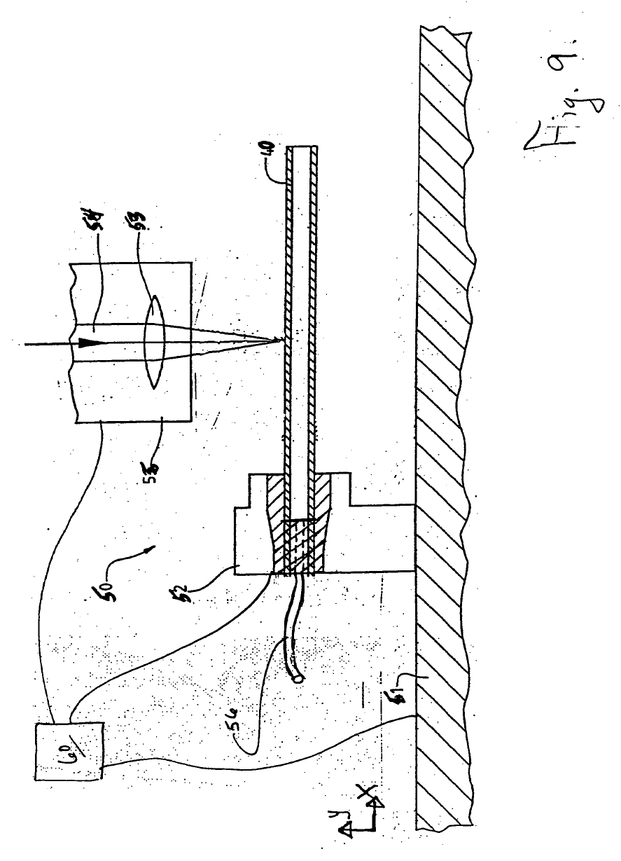

Other methods of fabricating the mechanically interlocked stent 10 according to the invention comprise separately cutting the male and female components 31, 32 along with the connected strut section 20, as seen in FIG. 14, and thereafter, separately assembling the male and female components 31, 32 to comprise the stent 10. Where the male and female components 31, 32 are separately formed, conventional laser cutting methods may also be used as described above. As the artisan will readily appreciate, the laser cutting methods may be on-center methods generally similar to as described above, or may be off-center or rotational methods permitting increased angular dimensions of the various components to comprise the mechanically interlocked stent according to the systems and methods of the invention. FIG. 10, also discussed further below, shows an example of an off-center laser cutting system 100 in which the laser head 110 is not fixed but instead is mounted to an x stage 101, a y stage 102, and a z stage 103 such that the laser head 110 can move transversely (x-stage) across the outside of the tubing and vertically (z-stage) to keep the laser beam focused.

Further, where the male and female components 31, 32 are separately formed and thereafter assembled, the male component may be made slightly larger or the female component slightly smaller due to the omission of the laser beam kerf width that otherwise accompanies the simultaneous formation of the male and female components as in earlier described embodiments. The slightly larger formation of the male component 31, or the smaller formation of the interior of the female component 32, when separately formed, provides an even tighter fit of the male components 31 within the female components 32 in which they are retained, while still permitting the intended rotational movement of the male components even as retained within the corresponding female component 32. Shape memory materials, such as nitinol, are preferred as the material that comprises the mechanically interlocked stent 10 when the male and female components 31, 32 are separately formed and thereafter assembled, although other materials can also be used as described elsewhere herein.

FIG. 10 illustrates an apparatus and method for fabricating a mechanically interlocked stent 10 according to the invention. According to the apparatus and method shown in FIG. 10, the various components comprising the stent 10 are laser cut using an off-center laser cutting approach, such as system 100. As a result, increased included angular dimensions of the male and female components 31, 32 may be achieved to increase the interference between the male and female component 31, 32 without changing the size of the male component, the thickness of tubing, or other attributes. Meanwhile, the rotational movement of the male component is generally unrestricted. As before, modifying either the kerf width (kw), material thickness (t) or other dimensions of the male and female components 31, 32 can modify the amount of interference between a male component 31 and a female component 32.

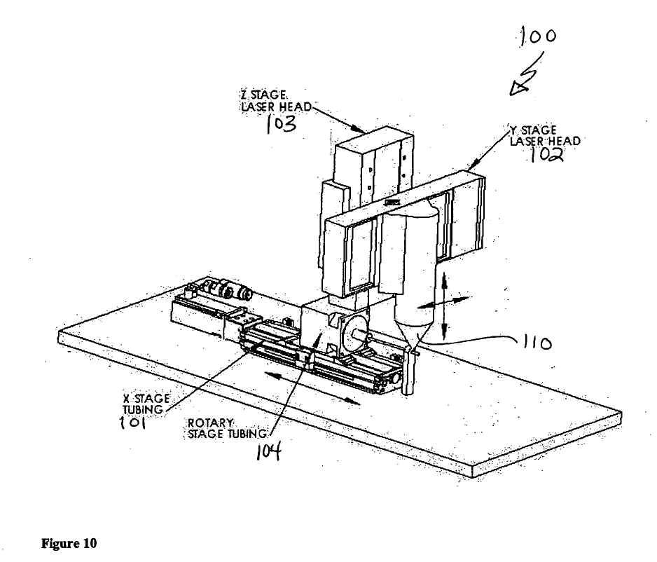

FIG. 11 shows a cross-sectional view of a stent 10 being cut by a standard laser system 200. The image illustrates the resulting wedge shaped cross-sectional geometry when tubes are cut by a standard laser system in which the laser head is fixed over the top dead center of the tube. It can be noticed that the laser beam is directed toward the center of the tube and once the tube is rotated and cut again, a wedge-shaped feature results.

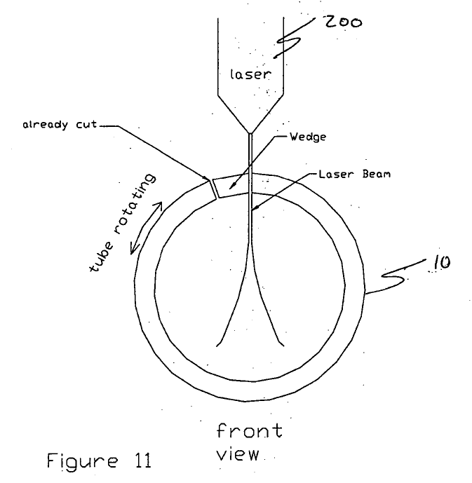

FIG. 12 illusrates isometrically mechanically interlocked stent 10 in cross-section. The wedge-shaped geometry of the mechanical interlock and the relationship of the male component 31 and female component 32 are evident in FIG. 12.

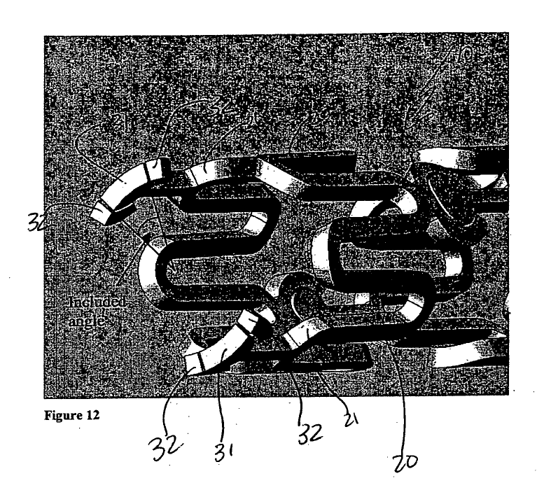

FIG. 13 is an isometric view of a mechanically interlocked stent 10 that has been cross-sectioned through a diametrically opposed set 25 of mechanical interlocks. The image shows how the mechanical interlocks of male and female components 31, 32, respectively, remain intact even if an external force is applied. If a male portion of an interlock is pushed or forced outward, the diametrically opposed male will limit that outward motion because its subsequent movement is inward which is limited due to the interference.

FIGS. 15a and 15b show respective cross-sectional views of a stent 10 being cut by a laser that has off-center cutting capabilities (FIG. 10). Off-center cutting allows the laser head to move transversely to the tube and vertically to keep the laser focused while the tube is moving axially and rotating under the laser beam. This ability to cut off-center allows the designer to increase the included angle of the male and female components without changing other attributes such as wall thickness or tubing OD. As previously mentioned, an increase in the included angle is desirable in order to increase the amount of inward interference between the male and female components. FIG. 15a shows the laser 110 cutting one side 31c of the male component 31 and FIG. 15b shows the laser 110 cutting the other side 31c of the male component 31. Since the laser 110 has traveled to the other side of the tube during the cutting process, the included angle α has increased because now the intersection of each cut path is no longer in the center of the tube as with a conventional laser system. During off-center cutting, the linear stage 101 and rotary stage 104 of the tubing moves in sync with the horizontal stage 102 and vertical stage 103 of the laser head.

The various exemplary embodiments of the invention as described hereinabove do not limit different embodiments of the present invention. The material described herein is not limited to the materials, designs, or shapes referenced herein for illustrative purposes only, and may comprise various other materials, designs or shapes suitable for the systems and procedures described herein as should be appreciated by one of ordinary skill in the art. For example, the mechanical interlock could be placed at different or additional locations on the stent in order to achieve the same or similar mechanical interlocking of the strut sections in accordance with the invention. Likewise, as the artisan will appreciate, the male component need not be limited to a ball shape with a correspondingly shaped female component for receipt thereof. Rather, the male component could be oblong, hexagonal, or otherwise shaped provided the female component is appropriately correspondingly shaped for receipt thereof in accordance with the systems and methods described herein.

While there has been shown and described what is considered to be preferred embodiments of the invention, it will, of course, be understood that various modifications and changes in form or detail could readily be made without departing from the spirit or scope of the invention. It is therefore intended that the invention be not limited to the exact forms described and illustrated herein, but should be construed to cover all modifications that may fall within the scope of the appended claims.

What is claimed is:

1. A cylindrical stent deployable in an anatomical system, the stent comprising:
a first end;
a second end;
an intermediate section between the first end and the second ends;
a longitudinal axis extending within the stent from the first end to the second end;
a series of longitudinally adjacent strut sections; and
a mechanical interlock joining designated pairs of the longitudinally adjacent strut sections.

2. The cylindrical stent of claim 1, wherein two or more designated pairs of the mechanically interlocked longitudinally adjacent strut sections comprise a set of mechanically interlocked strut sections.

3. The cylindrical stent of claim 2, further comprising neighboring sets of mechanically interlocked strut sections.

4. The cylindrical stent of claim 3, wherein each neighboring set is out of phase with one another.

5. The cylindrical stent of claim 4, wherein each mechanically interlocked strut section of the designated pair of mechanically interlocked strut sections is diametrically opposed to one another, and each set is 90° out of phase with neighboring sets thereof, the diametrically opposed relationship between the mechanical interlock pairs keeping the mechanical interlocks together.

6. The cylindrical stent of claim 4, wherein each mechanically interlocked strut section of the designated pair of mechanically interlocked strut sections is other than diametrically opposed to one another, and each set is other than 90° out of phase with neighboring sets thereof.

7. The cylindrical stent of claim 2, wherein the mechanical interlock is further comprised of a male component and a female component, the male component extending from a closed end of one of the designated pairs of longitudinally adjacent strut sections and the female component extending from a closed end of a corresponding one of the designated pair of longitudinally adjacent strut sections.

8. The cylindrical stent of claim 7, wherein the male component further comprises a shaft portion extending from the closed end of the one of the adjacent strut sections, and a ball with angled side surfaces extending therefrom, the female component receiving the ball.

9. The cylindrical stent of claim 8, wherein the female component is further comprised of a shaft, a receptacle, and angled side surfaces, inward movement of the ball of the male component being restrained by the angled side surfaces of the male and female components, while rotational movement of the male component is unimpeded.

10. The cylindrical stent of claim 9, wherein the interference between the male component and female component varies according to included angular dimensions of the side surfaces of the male and female components, kerf width and a thickness of the stent.

11. The cylindrical stent of claim 9, wherein the male and female components are laser cut from a same tube as comprises the stent.

12. The cylindrical stent of claim 2, wherein the stent is comprised of a bio-compatible material.

13. The cylindrical stent of claim 2, wherein the male components and the female components are simultaneously laser cut from a same tube as comprises the stent requiring no further assembly of the mechanical interlocks of the stent.

14. The cylindrical stent of claim 9, wherein the male components and the female components are separately laser cut from a same tube as comprises the stent and require further assembly to comprise the mechanical interlocks of the stent.

15. The cylindrical stent of claim 14, wherein the stent and male and female components are comprised of shape memory material.

16. A method of making a cylindrical stent with mechanically interlocked strut sections, the method comprising:
providing a tube of bio-compatible material;
laser cutting a series of longitudinally adjacent strut sections from the tube;
laser cutting male components from closed ends of some of the longitudinally adjacent strut sections of the tube; and
laser cutting female components from closed ends of longitudinally adjacent strut sections of the tube opposite a corresponding one of the male components, wherein the male components and the female components comprise designated pairs of longitudinally adjacent strut sections having a mechanical interlock.

17. The method of claim 16, further comprising simultaneously laser cutting the male and female components such that no further assembly of the mechanical interlock or stent is required.

18. The method of claim 16, wherein the strut sections, male components and female components are separately cut such that further assembly of the male component with a corresponding one of the female components is required to mechanically interlock the designated pairs of longitudinally adjacent strut sections of the stent.

19. The method of claim 17, wherein the stent, including the longitudinally adjacent strut sections, the male components and the female components are laser cut using on-center laser cutting.

20. The method of claim 17, wherein the stent, including the longitudinally adjacent strut sections, the male components and the female components are laser cut using off-center or rotational laser cutting.

21. The method of claim 18, wherein the stent, including the longitudinally adjacent strut sections, the male components and the female components are laser cut using on-center laser cutting.

22. The method of claim 18, wherein the stent, including the longitudinally adjacent strut sections, the male components and the female components are laser cut using off-center laser cutting.

23. The method of claim 16, wherein laser cutting the strut sections further comprises cutting longitudinally adjacent strut sections having undulating waves and closed ends, wherein two or more of the designated pairs of the closed ends of longitudinally adjacent strut sections that are mechanically interlocked comprise a set in which the designated pairs are cut to be in diametric opposition to one another within the set.

24. The method of claim 23, further comprising laser cutting neighboring sets of designated pairs of longitudinally adjacent strut sections such that neighboring sets are out of phase with one another.

25. The method of claim 23, wherein laser cutting the male components further comprises cutting a shaft section extending integrally from the designated pairs of closed ends of the strut sections, and cutting a ball having angled side surfaces integrally extending from each shaft.

26. The method of claim 25, wherein laser cutting the female components further comprises cutting a shaft section extending integrally from a corresponding one of the designated pairs of closed ends of the strut sections, cutting a receptacle extending integrally from each shaft, and cutting the receptacle at the same time as the male component with one pass of the laser beam, such that an interference fit between corresponding ones of the male components and the female components is sufficient to restrict inward movement of the ball of the male component while permitting rotational movement thereof within the female component.

27. The method of claim 22, wherein the designated pairs of closed ends are cut to be diametrically opposed to one another, and neighboring sets of diametrically opposed designated pairs of closed ends are cut to be out of phase 90° relative to one another.

28. The method of claim 22, wherein the designated pairs of closed ends are cut to be other than diametrically opposed to one another, and neighboring sets of the other than diametrically opposed designated pairs of closed ends are cut to be out of phase other than 90° relative to one another.

29. The method of claim 16, further comprising varying the angular or other dimensions of the male components, the female components, a kerf width of material removed by the laser, or a thickness of the stent to control the amount of inward movement of the male component within a corresponding female component, while permitting unimpeded rotational movement thereof.
